(19) United States
(12) Patent Application Publication
(10) Pub. No.: US 2016/0245271 A1
(43) Pub. Date: Aug. 25, 2016

(54) PERISTALTIC PUMP COMPRISING ANGULARLY VARIABLE PRESSURE ROLLERS

(71) Applicant: B. BRAUN AVITUM AG, Melsungen (DE)

(72) Inventors: Oliver SCHAEFER, Neuenstein (DE); Kai-Uwe RITTER, Rednitzhembach (DE)

(21) Appl. No.: 15/017,927

(22) Filed: Feb. 8, 2016

(30) Foreign Application Priority Data
Feb. 25, 2015 (DE) 10 2015 102 659.7

(51) Int. Cl. F04B 43/12; A61M 1/16; A61M 1/26; F04B 49/20; F04B 51/00

(52) U.S. Cl. CPC F04B 43/1253; F04B 49/20; F04B 51/00; A61M 1/267; A61M 1/1603

(57) ABSTRACT

A method of conveying fluid in an apparatus for extracorporeal blood treatment is disclosed, wherein fluid is conveyed by a peristaltic pump from a low-pressure side to a high-pressure side, an elastically deformable fluid line arranged between the low-pressure side and the high-pressure side being deformed between a support surface and a rotor rotating with respect to the latter and having at least two pinch elements, wherein the pinch elements are repositioned at angles relative to each other during rotation of the rotor for causing pre-compression. A dialysis machine including a peristaltic pump conveying fluid from a low-pressure side to a high-pressure side, wherein the peristaltic pump is arranged to receive an elastically deformable fluid line between the low-pressure side and the high-pressure side and includes a support surface supporting the fluid line and a rotor is also disclosed, the rotor including at least two pinch elements each deforming the fluid line between itself and the support surface, wherein the pinch elements are formed to be repositioned at angles relative to each other in the direction of rotation.

### CROSS REFERENCE TO RELATED APPLICATIONS

This application claims priority to German Application No. DE 10 2015 102 659.7 filed Feb. 25, 2015, the contents of such application being incorporated by reference herein.

### FIELD OF THE INVENTION

The invention relates to a method of conveying fluid, especially blood, in an apparatus for extracorporeal blood treatment, especially in a dialysis machine, wherein fluid is conveyed from a low-pressure side to a high-pressure side with a peristaltic pump and an elastically deformable fluid line arranged between the low-pressure side and the high-pressure side is deformed, especially pinched, between a support surface and a rotor rotating with respect to the same and having at least two pinch elements. Moreover, it relates to a dialysis machine comprising a peristaltic pump conveying fluid from a low-pressure side to a high-pressure side, the peristaltic pump including an elastically deformable fluid line between the low-pressure side and the high-pressure side, a support surface supporting the fluid line and a rotor, wherein the rotor includes at least two pinch elements, especially squeezing pinch elements, each deforming the fluid line between itself and the supporting surface.

### DESCRIPTION OF THE RELATED ART

Methods and dialysis machines of this type are known from the state of the art. The peristaltic pump of such system is intended to convey a defined volume of a medium such as blood or dialysis fluid by deforming and pinching off the elastically deformable fluid line. A peristaltic pump for conveying blood usually conveys from a negative pressure side (low-pressure side) to a positive pressure side (high-pressure side). Known systems in medical apparatuses for extracorporeal blood treatment usually consist of a rotor, a pump casing and a tube line which is arranged there between and convey a defined volume at a steady, i.e. constant pressure from the low-pressure side to the high-pressure side. In the case of a peristaltic pump including two pinch elements, they are arranged at a position of 180° steadily or, respectively, at a fixed angle of 180° relative to each other.

It is a drawback which unfortunately is frequently occurring with known dialysis machines and methods that during a pumping operation in the conveyed fluid volume undesired pulsation may occur on the high-pressure side. This detrimental effect is due to the fact that on the low-pressure side a volume section of the elastic fluid line is pinched off and the fluid volume enclosed therein is conveyed in the direction of the high-pressure side by rotation of the rotor and displacement of the pinching position of the fluid line caused thereby. When the conveying volume section is pinched off, the volume enclosed therein is under low-pressure side pressure. It is conveyed under said pressure to the high-pressure side, where high pressure is prevailing. If within the scope of the conveying operation the conveying volume section is opened toward the high-pressure side, due to the pressure difference between the high-pressure side and the conveying volume section a fluid flow takes place from the high-pressure side into the conveying volume section until pressure compensation is provided. As a consequence, the high-pressure side pressure briefly drops and pulsation occurs on the high-pressure side.

Another detrimental effect in known methods and dialysis machines resides in the fact that in the case of conveying blood as a fluid during the afore-described pressure compensation blood is squeezed through the opening bottleneck between the conveying volume section and the high-pressure side. As a rule, partial destruction of blood cells is occurring, which in general is referred to as hemolysis.

### SUMMARY OF THE INVENTION

Based on the afore-described state of the art, an object underlying the present invention is to eliminate the afore-listed drawbacks, especially to minimize the afore-described negative pulsation effect.

This object is achieved by the features of the independent claims.

According to aspects of the invention, this object is achieved by a method of conveying fluid in an apparatus for extracorporeal blood treatment, with fluid being conveyed from a low-pressure side to a high-pressure side with a peristaltic pump, wherein an elastically deformable fluid line arranged between the low-pressure side and the high-pressure side is deformed between a support surface and a rotor rotating vis-à-vis the same and having at least two pinch elements (or pressure rollers), in which method the pinch elements are angularly positioned relative to each other during rotation of the rotor for causing pre-compression. Furthermore, the object is achieved by a dialysis machine comprising a peristaltic pump conveying fluid from a low-pressure side to a high-pressure side, the peristaltic pump including an elastically deformable fluid line between the low-pressure side and the high-pressure side, a supporting surface supporting the fluid line and a rotor, wherein the rotor comprises at least two pinch elements each deforming the fluid line between itself and the running surface, wherein the pinch elements are formed to be angularly positioned relative to each other in the direction of rotation.

According to aspects of the invention, pre-compression of a fluid volume section conveyed from the low-pressure side to the high-pressure side is performed during conveying. The rotor and the support surface supporting the elastic fluid line are configured and adapted to each other so that a conveying path is formed there between. In the area of the conveying path the fluid line is deformed, especially pinched or pinched in a fluid-tight manner, between the support surface and a pinch element transversely to the cross-section thereof. A leading pinch element does not run out of the conveying path before a trailing pinch element has run into the conveying path. In other words, the angle between running into the conveying path and running out of the conveying path is larger than the angle between a leading pinch element and a trailing pinch element. Therefore, according to aspects of the invention, there is always a period of time and a conveying path section, respectively, in which a fluid volume conveyed between the leading pinch element and the trailing pinch element is enclosed there between. This period of time and, respectively, this conveying path section is used, according to aspects of the invention, to pre-compress the conveyed fluid volume so that pressure variations on the high-pressure side are minimized and preferably eliminated. In other words, by geometric dependencies the volume taken from the negative pressure side is compressed until the volume is given off on the positive pressure side and thus pulsation is minimized.

According to aspects of the invention, the rotor includes at least two pinch elements. This is the lowest possible number of pinch elements which is required for forming a defined, especially a sealed conveying path. It is within the scope of the invention when the rotor includes more than two pinch elements, in particular three or four. The angular positions of the pinch elements relative to each other, i.e. the angles formed between neighboring pinch elements, outside the area of pre-compression amount to 180° preferably in the case of two pinch elements, to 120° in the case of three pinch elements and to 90° in the case of four pinch elements. The length of the conveying path of the fluid line is larger in all cases.

The pre-compression is performed by reducing the angular distance of neighboring pinch elements, i.e. the angle between a leading pinch element and a pinch element trailing thereto, as long as both pinch elements are provided in the conveying path section and hence the latter is closed on both sides by the two pinch elements. A reduction of the distance of the respective pinch elements results in a reduction of the volume of the conveying path section. Since no fluid can escape due to the sealing of the latter with the pinch elements until the leading pinch element runs out of the conveying path section, an increase in pressure is resulting. The reduction of the distance of the respective pinch elements is selected so that a difference in pressure between the conveying path section and the high-pressure side is reduced and preferably balanced.

The pinch elements may be formed directly at the rotor, in particular integrally with the rotor. As an alternative, they may be arranged on rotor arms. These are preferably configured to be pivoting vis-à-vis the rotor in circumferential direction so that the pre-compression may be achieved via pivoting in the circumferential direction. The pinch elements can especially be in the form of pinch rollers or pressure rollers advantageously rolling off the fluid line in a material-saving manner or in the form of slide shoes that are slidingly moving over the fluid line.

The invention is adapted to achieve especially the following advantages:
reduction and, respectively, prevention of blood-damaging hemolysis, as reflux of fluid from the high-pressure area into the conveying path is reduced or prevented due to pressure difference,
pressure compensation between the low-pressure side and the high-pressure side due to the afore-described pre-compression,
reduction or even prevention of pulsation during the pumping operation.

Advantageous embodiments of the invention are claimed in the subclaims and shall be explained in detail hereinafter.

In an embodiment the fluid volume provided in the conveying path section is compressed by presetting the trailing pinch element in the direction of the leading pinch element. As a consequence, the volume enclosed between the two pinch elements is reduced and the fluid provided therein is pre-compressed. In other words, after enclosing the fluid volume to be conveyed the trailing pinch element rotates more quickly than the leading pinch element about the rotor axis for a particular period of time, until the desired pre-compression is reached.

In a different alternative embodiment, it may be provided that the leading pinch element is reset in the direction of the trailing pinch element. As a result, the volume enclosed between the two pinch elements is equally reduced and the fluid provided therein is pre-compressed. In other words, after enclosing the fluid volume to be conveyed the leading pinch element rotates more slowly than the trailing pinch element about the rotor axis for a particular period of time or stops (for a short time), until the desired pre-compression is reached. In addition, a combination of the two afore-mentioned embodiments is within the scope of the invention.

According to an embodiment of the invention, the pressure on the low-pressure side and the pressure on the high-pressure side are sensed and a pressure difference is formed. The angular positioning of the pinch elements relative to each other may then be performed depending on said pressure difference. Such pressure sensing is advantageously simple, as the pressures on the high-pressure and low-pressure side can be easily measured due to proper accessibility.

In another embodiment of the invention, the high-pressure side pressure and the pressure in the conveyed fluid volume are sensed and a pressure difference is formed therefrom. The pinch elements are angularly positioned relative to each other depending on said pressure difference. In this embodiment it is especially advantageous that by virtue of detecting the pressure in the conveying path section pre-compression and thus pressure compensation can be performed with special accuracy.

In a further embodiment the high-pressure side pressure pattern can be detected and the pinch elements can be angularly positioned relative to each other depending on the high-pressure side pressure pattern. It is advantageous in this case that pressure sensing has to be performed at one point in the system only, thus allowing the system to be designed in an especially simple and robust manner.

For pressure sensing in any one of the afore-described manners the dialysis machine may further include a pressure gauge for determining the inlet side pressure and/or a pressure gauge for determining the outlet side pressure and/or a pressure gauge for determining the pressure in the pump segment, i.e. in the conveying path formed in the fluid line.

In a mode of the invention, the pinch element running out of the conveying path can be transferred to a neutral position relative to the leading pinch element after running out of the conveying path section. In this way an especially easy control and setting, respectively, of the pre-compression is enabled.

It is of particular advantage when the pinch elements interact with a curve actuator, especially a curve or cam disk or a curve or cam shaft, the angular position of the pinch elements relative to each other being adjustable with the curve actuator. The pre-compression can be adjusted in a very simple and reproducible way by varying the curve geometry. Alternatively, each pinch element may be driven with a drive unit, especially with a step motor. This offers the advantage that a setting or variation of the pre-compression is especially easy to control.

In other words, the invention relates to a pressure-compensating rotor which is part of a peristaltic (tube) reel pump, especially a peristaltic pump for medical engineering the intended use of which is in extracorporeal blood treatment. Said rotor enables, together with the elastic material properties of the pump segment of a transfer system which is inserted in loops against a cylindrical running surface of the pump casing, a pump function which ensures blood transport to a dialyser. It can also be said that the present invention achieves the underlying object in that the rotor varies the position of the pinch elements relative to each other for several times within one revolution (360°). This means that in the case of a rotor having two pinch elements, for example, the latter take a relative position of 180° when taking up the volume on the negative pressure side. After the volume is enclosed by the second pinch element, the position of the pinch elements relative to each other varies to less than 180°. In this way the pressure of the volume enclosed in the conveying section of the fluid line is increased. Ideally the increase in pressure is interpreted so that the pressure in the enclosed segment preferably approaches the pressure on the pump output side. After giving off the volume on the positive pressure side, the position of the pinch elements relative to each other can vary to 180° again. These cyclic changes of position of the pinch elements can be transmitted, for example by a geometrically defined contour, e.g. a curve disk or cam geometry, to a rotor which is split and rotatably supported, for instance.

It is especially within the scope of the invention that the rotor includes two arms each supporting one pinch element. The latter may be driven especially individually, for example by a step motor, so that their position relative to each other can be freely controlled. When the input-side and output-side pressure is measured, as it is also common today, it is of advantage to render the advance angle of the second roller adjustable depending on the pressure difference. It is the target to minimize or even extinguish the pulsation. Furthermore, the pulsation can be established on the output side with a pressure sensor and the advance angle can be regulated to minimum pulsation. At this point of operation then the minimum hemolysis does occur.

Another aspect of the invention relates to a control means for a dialysis machine including a peristaltic pump which conveys fluid from a low-pressure side to a high-pressure side, the peristaltic pump comprising an elastically deformable fluid line between the low-pressure side and the high-pressure side, a support surface supporting the fluid line and a rotor, wherein the rotor includes at least two pinch elements each deforming the fluid line between itself and the support surface, wherein each pinch element is driven with a drive unit, especially with a step motor, and wherein the dialysis machine comprises a pressure gauge for determining the inlet-side pressure and/or a pressure gauge for determining the outlet-side pressure and/or a pressure gauge for determining the pressure in the fluid line. In accordance with the invention, the control means controls at least one drive unit for causing pre-compression so that the relative angle is varied, and especially reduced, in the direction of rotation between the pinch elements on the basis of the determined inlet-side pressure and/or the determined outlet-side pressure and/or the determined pressure in the fluid line.

### BRIEF DESCRIPTION OF THE DRAWINGS

The invention is best understood from the following detailed description when read in connection with the accompanying drawings. Included in the drawings are the following figures:

FIG. 1 shows a schematic of a cutout of an apparatus for extracorporeal blood treatment in an exemplary embodiment,

FIG. 2 shows an exemplary schematic of a control path in accordance with the invention,

FIG. 3 is a schematic top view onto a peristaltic pump according to aspects of the invention in a first state at a first time of operation,

FIG. 4 is a schematic top view onto the peristaltic pump of FIG. 3 in a second state following the first state at a second time of operation,

FIG. 5 is a schematic top view onto the peristaltic pump of FIG. 3 in a third state following the second state at a third time of operation and

FIG. 6 is a schematic top view onto the peristaltic pump of FIG. 3 in a fourth state following the third state at a fourth time of operation.

### DETAILED DESCRIPTION OF THE FIGURES

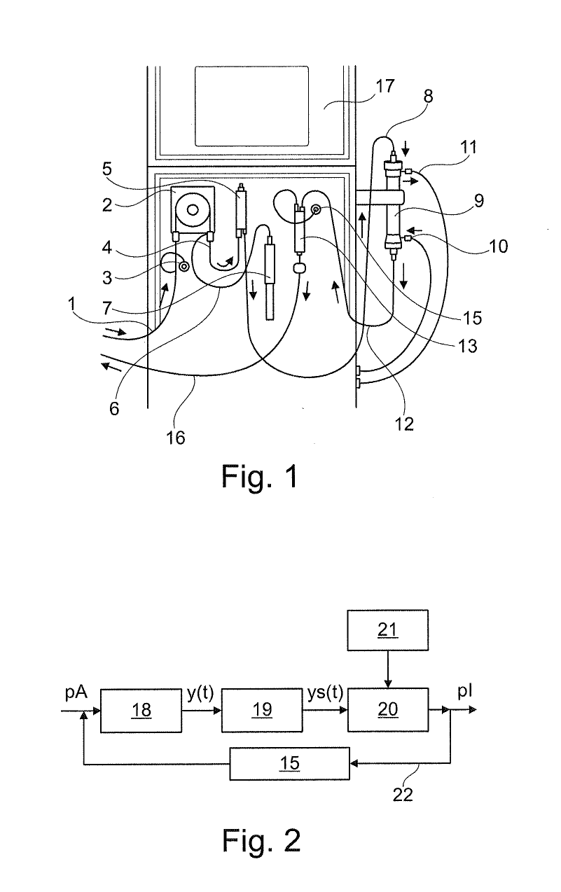

FIG. 1 exemplifies a cutout of an apparatus for extracorporeal blood treatment according to aspects of the invention. Here substantially the entire extracorporeal blood circuit of the apparatus is shown. It includes an arterial blood line 1 with which blood is guided from a patient (not shown) to a peristaltic pump 2 of the treatment apparatus. Upstream of the peristaltic pump 2 an arterial pressure sensor 3 is provided by which the pressure upstream of the peristaltic pump 2, i.e. the low-pressure side pressure, is measured. On the high-pressure side of the peristaltic pump 2 a high-pressure blood line 4 leads to an arterial air trap 5. Directly at the outlet of the peristaltic pump 2 additives can be added to the blood provided in the system with a feed line 6 and a pump 7, e.g. heparin for hemodilution.

From the arterial air trap 5 a line 8 guides blood which is under high pressure but not yet treated to a dialyser 9. On the input side dialysis fluid is supplied to the latter via a dialysis fluid feed line 10. In the dialyser 9 blood is treated, e.g. purified, in a known way with the dialysis fluid. Used dialysis fluid is removed from the dialyser 9 through a dialysis fluid drain 11 and is supplied to proper disposal or recycling (not shown). Treated blood is guided with a blood drain 12 from the dialyser 9 to a venous air trap 13 and is precipitated by the air of the latter. At the venous air trap 13 a venous pressure sensor 15 is provided by which the venous pressure, i.e. the high-pressure side pressure, is sensed. Treated blood is guided from the venous air trap 13 back to the patient via a venous blood line 16. FIG. 1 also shows a unit 17 for monitoring and controlling the apparatus.

The monitoring unit 17 serves, inter alia, for implementing the control loop shown in FIG. 2. The variable pA to be controlled is the set pressure at the output of the blood pump 2, hence the pressure in the high-pressure blood line 4. With a controller 18 an actuating variable y(t) of a step motor 19 is influenced and is introduced to a control path 20 as ys(t). On the latter disturbance variables 21 are acting, e.g. in the form of varying temperature or a slowly progressing ageing of the lines of the apparatus. From the control path the actual variable pl, i.e. the actual pressure at the output of the blood pump 2, is resulting. The latter is sensed by the pressure sensor 15 and is introduced to the control path again via a return line 22. It should be noted that the pressure sensor 15 or an additional pressure sensor, other than exemplified in FIG. 1, may be provided directly downstream of the peristaltic pump 2.

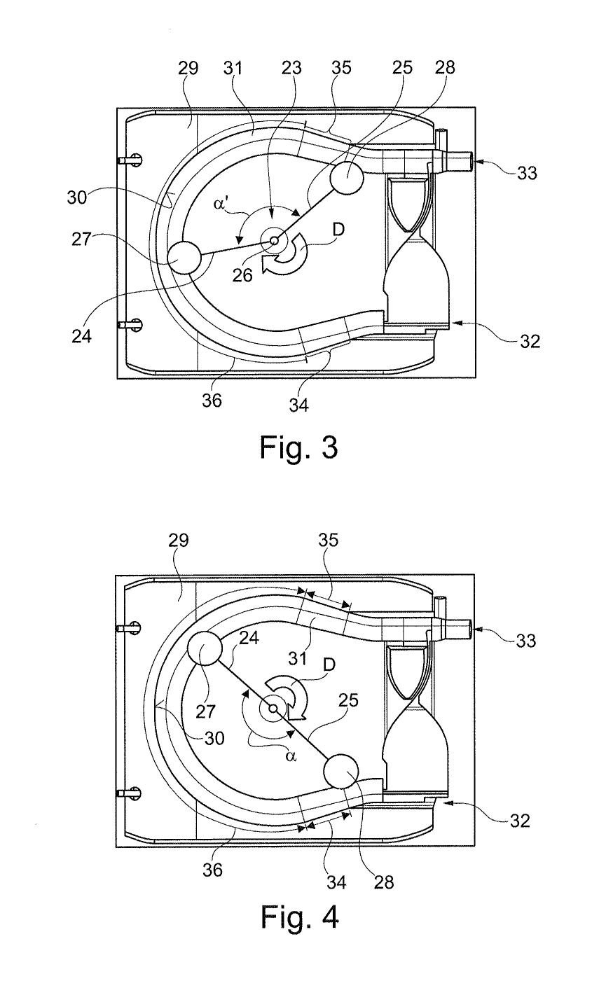

FIGS. 3 to 6 illustrate the peristaltic pump 2 including the arterial blood line 1 and the high-pressure blood line 4 in a cutout at different points in time during the method according to aspects of the invention. The peristaltic pump 2 includes a rotor 23 comprising a first rotor arm 24 and a second rotor arm 25. The rotor arms 24, 25 rotate about a common rotor axis 26. The first rotor arm 24 supports on its side facing away from the rotor axis 26 a first pinch element 27 in the form of a first pinch roller 27. The second rotor arm 25 supports on its side facing away from the rotor axis 26 a second pinch element 28 in the form of a second pinch roller 28. Moreover, the peristaltic pump 2 comprises a blood pump casing 29 which in a known way forms a support surface 30. In the blood pump casing 29 a fluid line 31 is arranged such that it is deformed between the support surface 30 and the pinch elements 27, 28. It is especially elastically deformed in such way that its cross-section is partly squeezed, i.e. narrowed, and especially completely pinched, i.e. closed in a substantially fluid-tight manner.

The fluid line 31 is connected on the side of its inlet 32 to the arterial blood line 1 and on the side of its outlet 33 to the high-pressure blood line 4. The fluid line 31 is arranged in a subzone between an inlet section 34 and an outlet section 35 in the form of a pitch circle. The inlet section 34 reaches from the zone of the fluid line 31 in which the pinch elements 27, 28 enter into contact with the same, while the rotor 23 is rotating, to the zone of the fluid line 31 in which the deformation of the cross-section of the fluid line 31 by the pinch elements 27, 28 is completed. Inversely, the outlet section 35 reaches from the zone of the fluid line 31 in which the deformation of the cross-section of the fluid line 31 by the pinch elements 27, 28 is fully provided to the zone of the fluid line 31 in which the pinch elements 27, 28 lose the contact to the fluid line while the rotor is rotating. Between the inlet section 34 and the outlet section 35 the fluid line 31 forms a conveying path section 36. In FIG. 3 the conveying path section 36, the inlet section 34 and the outlet section 35 are marked regarding their extension over the respective angular area about the rotor axis 26.

FIG. 3 illustrates the peristaltic pump 2 shortly before the compressed conveying volume is discharged. This state shall be illustrated in detail hereinafter along with the state shown in FIG. 6.

FIG. 4 illustrates the peristaltic pump 2 at an operating time at which the pinch element 28 just runs into the inlet portion 34 and starts to compress the fluid line 31 and to narrow the cross-section thereof. The pinch element 27 is provided in the zone of the conveying path section 36 and pinches the fluid line 31 so that its cross-section is substantially completely closed at the position of engagement of the pinch element 27. In this configuration, the pinch element 27 forms the leading pinch element, while the pinch element 28 is the trailing pinch element. The angle α between the rotor arms 24, 25 (and thus between the pinch elements 27, 28) inserted in FIG. 4 in this state amounts to 180° and the pinch elements 27, 28 are in the so called neutral position relative to each other.

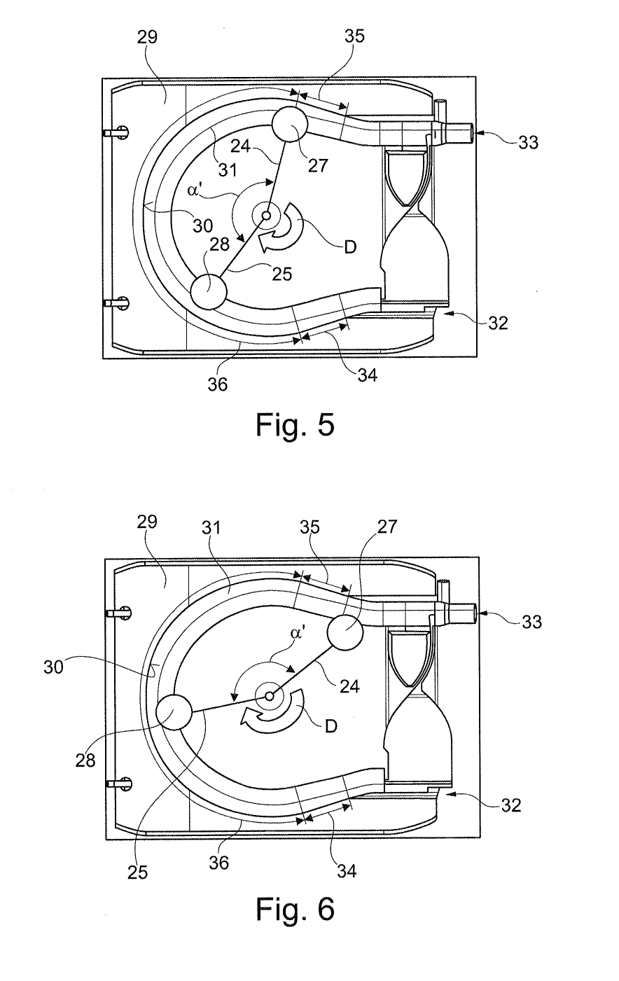

A somewhat later point in time is shown in FIG. 5. The rotor 23 has continued rotating in the indicated direction of rotation D. At the shown point in time the leading pinch element 27 is located exactly at the beginning of the run-out section 35 of the conveying path 36 and continues closing the cross-section of the fluid line 31 at the point of its engagement in a fluid-tight manner. The trailing pinch element 28 has further penetrated the conveying path section 36 and now equally closes the cross-section of the fluid line 31 at the point of engagement in a fluid-tight manner. Between the leading pinch element 27 and the trailing pinch element 28 a fluid volume or fluid conveying volume sealed on both sides by the engagement of the pinch elements 27, 28 is formed.

It is clearly evident from a comparison of FIG. 4 and FIG. 5 that during rotation from the state shown in FIG. 4 into the state shown in FIG. 5 the angle α between the two pinch elements has been diminished to the amount α′ (α′<α or α′+Δα=α). This is achieved in that either the trailing pinch element 28 rotates more quickly about the rotor axis 26 than the leading pinch element 27 over a particular angular range or period of time and/or in that the leading pinch element 27 rotates more slowly than the trailing pinch element 28 over a particular angular range or period of time and/or in that the leading pinch element 27 stops over a particular (short) period of time. The angular variation Δα causes a reduction of the conveying fluid volume enclosed between the two pinch elements 27, 28. Since, due to the fluid-tight sealing of the two ends of the conveying fluid volume with the pinch elements 27, 28, no fluid can escape from the conveying volume, the intended pre-compression takes place. The magnitude of the angular variation Δα is selected so that the pressure in the conveying path corresponds substantially to the pressure on the high-pressure side or is at least approximated at the best to the same.

In the course of the further rotation of the rotor 23 into the position shown in FIG. 6 in which the leading pinch element 27 is provided at the end of the run-out section 35, the fluid line 31 is opened in the zone of engagement of the pinch element 27 so that fluid may flow out of the conveying volume into the high-pressure side fluid line 4. Since in the conveying volume substantially the same pressure as in the high-pressure side blood line 4 is prevailing, upon opening the conveying volume section by releasing the pinch element 27 from the fluid line 31 no or only a small pressure variation will occur. The state shown in FIG. 6 corresponds to the state shown in FIG. 3 with the exception that the rotor 23 has been rotated about 180° and in FIG. 3 the pinch element 28 (instead of the pinch element 27) runs out of the run-out section 35. It is referred to the fact that in the course of further rotation from the state shown in FIG. 6 the angle between the pinch element 27 and the pinch element 28 again increases from the amount α′ to the amount α, in the illustrated example to 180°, and the pinch element 27 in such constellation runs into the run-in section 34 again and enters into contact with the fluid line 31. The afore-mentioned pre-compression taking place between the pinch elements 27 and 28 is repeated in the same way between the pinch elements 28 and 27.

1-12. (canceled)

13. A method of conveying fluid in an apparatus for extracorporeal blood treatment, comprising the steps of:
conveying fluid through an elastically deformable line with a peristaltic pump from a low-pressure side to a high-pressure side, the elastically deformable fluid line deformed between a support surface and a rotor rotating in a rotation direction relative to the support structure, the rotor having at least two pinch elements;
repositioning the pinch elements at angles relative to each other during rotation of the rotor in the rotation direction to cause pre-compression in the elastically deformable fluid line.

14. The method of claim 13, wherein between two neighboring pinch elements a fluid volume to be conveyed is enclosed and subsequently a volume compression of the enclosed fluid volume results from reduction in the relative angular distance a of the two neighboring pinch elements.

15. The method according to claim 13, wherein a trailing pinch element is repositioned in the rotation direction relative to a leading pinch element.

16. The method according to claim 13, further comprising:
determining a pressure difference between the low-pressure side and the high-pressure side;
wherein the repositioning of the pinch elements relative to each other is performed depending on pressure difference.

17. The method according to claim 13,
determining a pressure difference between the high-pressure side and line pressure in the elastically deformable fluid line;
wherein the repositioning of the pinch elements relative to each other is performed depending on pressure difference.

18. The method according to claim 13, wherein the high-pressure side pressure pattern is detected and the angular positioning of the pinch elements relative to each other is performed depending on the high-pressure side pressure pattern.
determining a pressure pattern in the high-pressure side;
wherein the repositioning of the pinch elements relative to each other is performed depending on pressure pattern.

19. The method according to claim 13, further comprising:
repositioning an exiting pinch element exiting a conveying path into a neutral position relative to a leading pinch element in the conveying path.

20. A dialysis machine comprising:
a peristaltic pump having a rotor, a low-pressure side, a high-pressure side, and a support arranged to support an elastically deformable fluid line, the peristaltic pump configured to convey fluid through the elastically deformable line from the low-pressure side to the high-pressure side;
the rotor having a direction of rotation and including at least two pinch elements, each pinch element configured to deform the fluid line between itself and the support surface, the pinch elements configured to be angularly repositioned relative to each other in the direction of rotation.

21. The dialysis machine of claim 20, wherein the pinch elements interact with a curve actuator to reposition the pinch elements relative to each other.

22. The dialysis machine of claim 21, wherein the curve actuator is a curve, cam disk, or cam shaft.

23. The dialysis machine of claim 20, wherein each pinch element is driven by a step motor.

24. The dialysis machine of claim 20, wherein the dialysis machine further comprises:
at least one of an inlet pressure gauge configured to determine the inlet-side pressure, an outlet pressure gauge configured to determine the outlet-side pressure, or a line pressure gauge configured to determine line pressure in the fluid line.

25. A controller for a dialysis machine having a peristaltic pump conveying fluid from a low-pressure side to a high-pressure side, the peristaltic pump arranged to receive an elastically deformable fluid line between the low-pressure side and the high-pressure side and the peristaltic pump including a support surface supporting the fluid line and a rotor, the rotor including at least two pinch elements, each pinch element arranged to deform the fluid line between itself and the support surface and driven by a drive unit;
the controller configured to control the drive unit such that the relative angle of the pinch elements are varied in the direction of rotation to cause pre-compression in the deformable fluid line.

26. The controller of claim 25, wherein the dialysis machine further includes at least one of an inlet pressure gauge for determining the inlet-side pressure, an outlet pressure gauge for determining the outlet-side pressure, or a line pressure gauge for determining line pressure the fluid line;
the controller configured to control the drive unit on the basis of the determined at least one of the inlet-side pressure, the outlet-side pressure, or the line pressure.
